(19) United States
(12) Patent Application Publication
(10) Pub. No.: US 2025/0262374 A1
(43) Pub. Date: Aug. 21, 2025

(54) FEEDING TUBE MANUAL PUMP

(71) Applicant: Carl A. OLSON, Lagrange Highlands, IL (US)

(72) Inventor: Carl A. OLSON, Lagrange Highlands, IL (US)

(21) Appl. No.: 19/058,383

(22) Filed: Feb. 20, 2025

Related U.S. Application Data
(60) Provisional application No. 63/555,751, filed on Feb. 20, 2024.

(51) Int. Cl. A61M 5/142; A61J 15/00; A61M 5/168

(52) U.S. Cl. CPC A61M 5/1424; A61J 15/0015; A61M 5/16813

(57) ABSTRACT

A pump for use in a system for tube feeding, the pump having a body portion having a manual plunger therein, and having a first opening with a first valve and a second opening with a second valve; wherein the first opening with the first valve is configured to permit flow of a liquid feed composition through the first opening into the body portion; wherein the second opening with the second valve is configured to permit flow of the liquid feed composition through the second opening out of the body portion, a system containing the pump and feed reservoir, and a method of feeding using the system.

### CROSS REFERENCE TO RELATED APPLICATIONS

The present application is related to and claims priority to U.S. Provisional Application Ser. No. 63/555,751, filed Feb. 20, 2024, the contents of which are incorporated herein by reference in their entirety.

### BACKGROUND

### Field of Invention

The present invention relates generally to feeding tubes, and more particularly to mechanisms of feed delivery via a feeding tube.

### Description of Related Art

Some individuals require a feeding tube to eat (enteral nutrition) due to various medical conditions or circumstances that impair their ability to consume food orally. One common reason is swallowing difficulties, known as dysphagia, which can result from neurological conditions including but not limited to stroke, cerebral palsy (CP) Parkinson's disease, or ALS. These difficulties make it unsafe or impossible to swallow without risking choking or aspirating food into the lungs. Additionally, some individuals may have conditions or injuries that prevent them from chewing or digesting food properly, such as severe facial trauma or gastrointestinal surgeries. In critical care settings, patients who are comatose, severely injured, or undergoing major surgeries may need feeding tubes to ensure they receive essential nutrition while unable to eat by mouth. Furthermore, individuals with chronic illnesses like advanced cancer or severe eating disorders may require feeding tubes to address malnutrition or inadequate nutritional intake. Feeding tubes serve not only to provide nutrition but also to administer medications when oral intake is not possible or insufficient to provide the dietary needs the individual requires.

There are several methods and mechanisms for feeding someone who has a feeding tube, depending on their specific needs and medical condition. One common method involves using gravity to deliver liquid nutrition through the feeding tube via a gravity drip. In this approach, the prescribed formula or liquid nutrition is poured into a feeding bag that is then kept at a higher elevation and attached to the feeding tube, allowing it to flow gradually via gravity into the stomach or small intestine. Gravity feeding is typically done while the individual is in an upright position to facilitate proper flow and digestion.

Another method of feeding through a feeding tube is bolus feeding, which involves administering the prescribed amount of formula or liquid nutrition directly into the tube using a syringe. Bolus feeding is usually done several times a day, with each feeding session lasting about 15-30 minutes. This method allows for more precise control over the feeding rate and volume, making it suitable for individuals with specific dietary requirements or medical conditions.

For individuals who require continuous or controlled feeding over an extended period, enteral feeding pumps are often used to deliver nutrition through the feeding tube at a predetermined rate. Feeding pumps can be programmed to administer the prescribed amount of formula or liquid nutrition at regular intervals throughout the day or night, ensuring a steady supply of nutrients and hydration. This approach is particularly beneficial for individuals who have difficulty tolerating large bolus feedings or who require precise control over their feeding regimen.

### SUMMARY

One object of the present invention is to provide a feeding pump system for improving quality of life for the patients and caretakers.

A further object of the present invention is to provide a feeding pump system convenient for everyday use that can be used anywhere where a feed would be administered.

Another object of the present invention is to provide a feeding pump system convenient for travel including but not limited to airports, in an airplane, or on a car ride.

An additional object of the present invention is to provide a feeding pump system that is inconspicuous and compact compared to the gravity feeding or a mechanical pump and doesn't require any outside power source or gravity (feed held at a higher elevation) to administer the feed.

A further object of the present invention to provide a feeding bag compatible with a feeding pump, to aid in priming the feeding pump, removing practicably all the air from a feeding pump system.

Another object of the present invention is to provide a method of administering feed using a feeding pump system.

These and other objects and advantages of the invention, either alone or in combinations thereof, may be satisfied by a pump for use in a system for tube feeding. The pump comprises a body (or barrel) portion having a manual plunger therein, and having a first opening with a first valve and a second opening with a second valve. The first opening with the first valve is configured to permit flow of a liquid feed (food, water, or medication) composition through the first opening into the body portion. The second opening with the second valve is configured to permit flow of the liquid feed composition through the second opening out of the body (or barrel) portion. The manual plunger is configured to draw the liquid feed composition through the first opening into the body (or barrel) portion when pulled to a withdrawn position, and to push the liquid feed composition through the second opening out of the body portion as the manual plunger is pushed into the body portion.

The foregoing and other features of the application are described below with reference to the drawings.

### BRIEF DESCRIPTION OF THE DRAWINGS

FIG. 1 shows an exemplary feeding pump system.

FIG. 2 shows an exemplary feed reservoir.

FIG. 3 shows another exemplary feed reservoir.

FIG. 4A shows an exemplary pump.

FIG. 4B shows a close-up of an entry end of another exemplary pump.

FIG. 5 shows an exploded view of the exemplary pump.

FIG. 6A shows a perspective view of the plunger and at least one diaphragm valve.

FIG. 6B shows a close-up cross-section view of the plunger and a pump.

FIG. 6C shows a detailed top view of the plunger and at least one diaphragm valve.

FIG. 6D shows a detailed bottom view of the plunger and at least one diaphragm valve.

FIG. 7 shows an exemplary feeding pump.

FIG. 8 shows an exemplary feeding pump system in a priming stage.

FIG. 9A shows the exemplary feeding pump in a first position.

FIG. 9B shows the exemplary feeding pump in a second position.

FIG. 10 shows an exemplary feeding pump system in a feeding stage.

FIG. 11 shows an exemplary feeding pump system in a cleaning stage.

### DETAILED DESCRIPTION

### Feeding Pump System

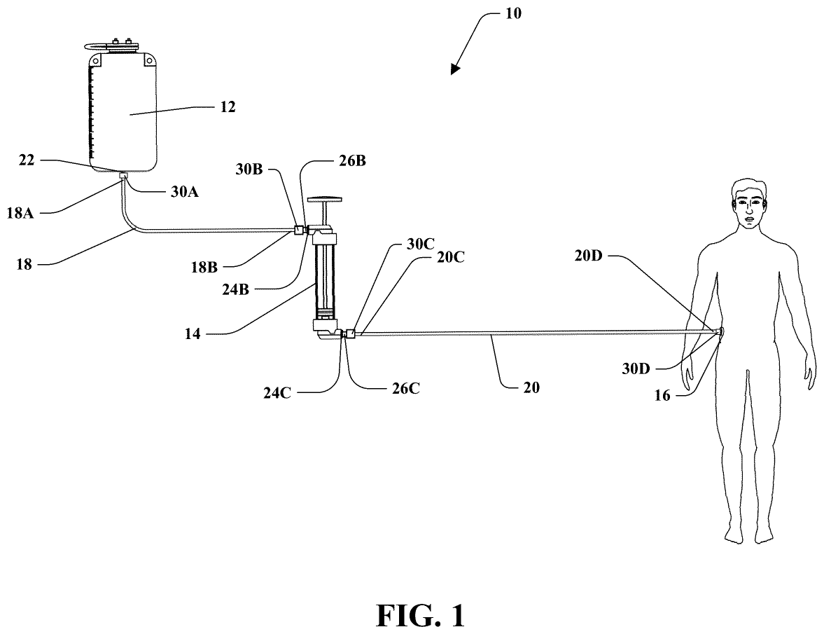

FIG. 1 shows an embodiment of a feeding pump system (10). The feeding pump system (10) is configured to deliver including but not limited to a liquid or semi-liquid feed composition to the patient. “Feed” may include enteral nutrition formulas, blended foods, medication and supplement solutions, hydration and electrolyte solutions, water, and other beverages. The viscosity of the feed may be a factor to contribute to the rate of feeding. The feeding pump system may comprise a feed reservoir (12) removably coupled to a pump (14) removably coupled to a feeding port (16) on a patient. The feeding pump system (10) may be similar to a manual bilge pump to administer feed from the feed reservoir (12) to the patient.

The feed reservoir (12) may be removably coupled to the pump (14) via an entry tube (18). “Tube” may include feeding tubes, where the diameter is typically measured in French Units (F), 1 French Unit=0.33 mm. Diameter of a tube may range from 5 F, to 36 F, where 18 F is approximately ¼ inch. The preferred tube diameter is 18 F; however, the tube diameter may vary based on patient needs and may be a controlling factor in the rate of feeding. It may be appreciated; that French Units are the current industry standard, but tubing diameter is not necessarily restricted to the described sizes. The entry tube (18) may have a first end (18A) and a second end (18B). The first end of the entry tube (18A) may be removably coupled to the feed reservoir (12) at an aperture (22) via a connector (30A). “Connector” may include ENFit® or an alternative medically-acceptable connector known in the art, preferably selected such that the connector of feeding tubes provides increased patient safety standards to ensure that feeding tube connectors are incompatible with the connectors for unrelated delivery systems such as trach tubes, IV lines, and catheters. The male and female connectors may be provided so the user may only be able to connect the components of the feeding pump system (10) in proper configuration, in order to ensure patient safety. In another embodiment, the first end of the entry tube (18A) may be fixedly attached or fully integrated into the feed reservoir (12) at the aperture (22). The second end of the entry tube (18B) may be removably coupled to the pump (14) at a first opening (24B) via a connector (30B). The first opening (24B) may contain a first valve (26B) configured such that feeding composition, air, or water may only pass through from the entry tube (18) into the pump (14).

The pump (14) may be removably coupled to the feeding port (16) via an exit tube (20). The feeding pump system (10) may be used with various types of feeding tubes and feeding ports, including but not limited to; enteral feeding, gastro-enteral tube, gastro-jejunostomy (GJ-tube), gastrostomy (G-tube), nasoduodenal (ND-tube), nasogastric (NG-tube), nasojejunal (NJ-tube), orogastric (OG-tube), or percutaneous endoscopic gastrostomy (PEG). The exit tube (20) may have a first end (20C) and a second end (20D). The first end of the exit tube (20C) may be removably coupled to the pump (14) at second opening (24C) via a connector (30C). The second opening (24C) may contain a second valve (26C) configured such that feeding composition, air, or water may only pass through from the pump (14) into the exit tube (20). In another embodiment, the first end of the exit tube (20C) may be fixedly attached or fully integrated into the pump (14) at the second opening (24C). The second end of the exit tube (20D) may be removably coupled to the feeding port (16) on a patient via a connector (30D). The valves (26B, 26C) may be one-way valves such that, for patient safety, the feeding pump system (10) cannot be used to evacuate contents from the patient's stomach. The feeding pump system (10) may only directionally deliver feed actuate by the pump (14) to flow from the feed reservoir (12) through the pump (14) and into the patient via the feeding port (16).

The feeding pump system (10) components may be made of any suitable material. It is preferable that the components may be recyclable and/or non-microbial materials. Non-microbial material is preferable in order to limit biological growth. The components' materials are preferably selected to comply with medical regulatory standards. Components may be opaque or optically clear for visual inspection. In another embodiment, the components may be primarily opaque with a transparent window to determine fill level. The pump (14) is preferably made of a clear material to allow visual inspection of feed composition as it travels through the system. Additionally, it is preferable that valves (26), tubing (18, 20), and connectors (30) are clear. It may be appreciated to those in the art that the components may alternatively be translucent or opaque, or any material of varying transparency.

### Feed Reservoir

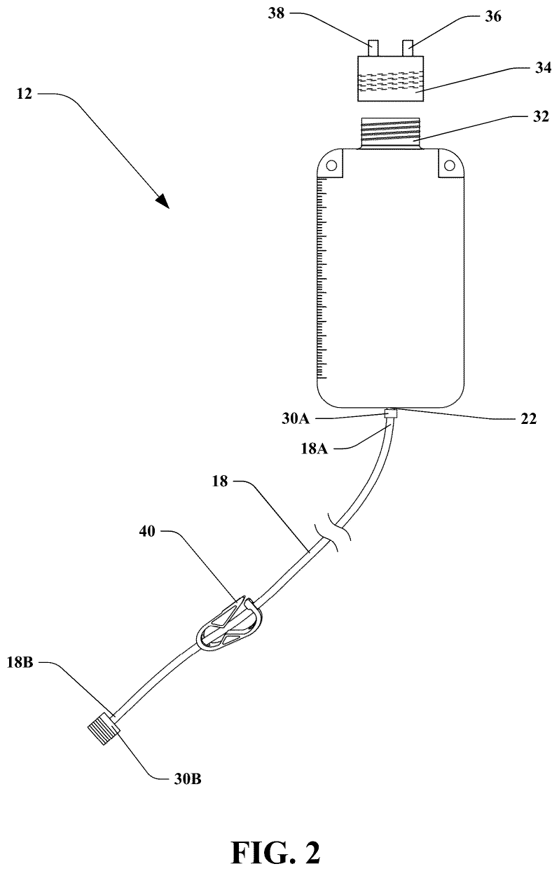

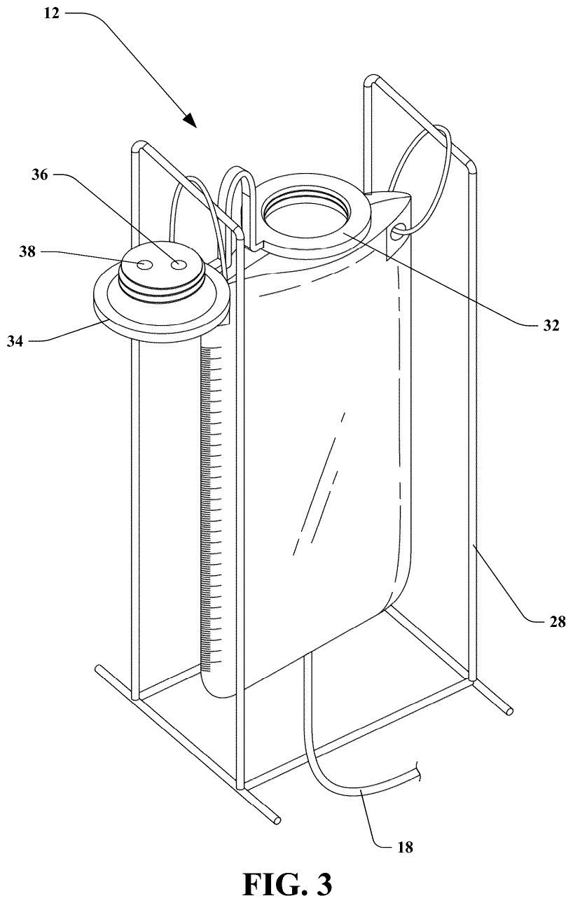

FIG. 2 shows an embodiment of a feed reservoir (12) and FIG. 3 shows another embodiment of the feed reservoir (12) with a frame (28). The feed reservoir (12) is preferably a single-use container, intended to be replaced daily. The feed reservoir (12) is preferably a bag; however, any suitable substantially airtight container may be conceived. The feed reservoir (12) may include markings or other appropriate methods of measuring the volume of feed in the feed reservoir. When the feed reservoir (12) is a bag, it can preferably be flattened and compacted for storage when unfilled. The feed reservoir (12) may have a configuration such that when at least partially filled, the feed reservoir takes on a shape with a flat bottom where the feed reservoir (12) may stand on a horizontal surface without external support. Furthermore, the feed reservoir configuration may include but is not limited to flat, pleated, or gusseted designs. In an alternative embodiment, the feed reservoir (12) may stand upright with the aid of a support structure or a frame (28), such that all apertures, openings, mouths, and the like are accessible while the feed reservoir (12) is seated in the frame (28). In an alternative embodiment, the feed reservoir (12) may include grommets in order to hang the feed reservoir (12) from the frame (28). In an alternative embodiment, the feed reservoir (12) may be a rigid container that may stand upright without any external force, filled or unfilled. The feed reservoir (12) having the capability of standing upright aids a user in dispensing feed into the feed reservoir (12). The feed reservoir (12) may also include hooks or loops to allow the feed reservoir (12) to be hung or attached to the patient, or a patient's wheelchair, a bed, or the like.

The feed reservoir (12) preferably has an opening for filling the feed reservoir (12), more preferably configured as a mouth (32) with a lid (34). The lid (34) may seal the mouth (32) to be substantially airtight. The lid (34) and mouth (32) may be threaded to create a screw-type sealed connection. In an alternative embodiment, the lid (34) may be a plug or stopper to seal the mouth (32). It may be appreciated by a person having skill in the art that the lid (34) and mouth (32) may have any suitable substantially airtight connection. A user may fill the feed reservoir (12) with a daily feed amount, depending on patient specifications, via the mouth (32) and replace the lid (34) to create a substantially airtight seal. In an alternative embodiment, the lid (34) may include a feedback port (36) and an air release valve (38). In another embodiment, the feedback port (36) and the air release valve (38) may be integrated into the wall of the feed reservoir (12), preferably near the top of the feed reservoir (12) or opposite the aperture (22), which serves as the exit from the feed reservoir (12). The first end of the entry tube (18A) may be removably coupled to the feed reservoir (12) at the aperture (22) via a connector (30A). In another embodiment, the first end of the entry tube (18A) may be fixedly attached or fully integrated into the feed reservoir (12) at the aperture (22). In another embodiment, there may be a means of cutting off flow (40) of feed through the entry tube (18). The means of cutting off flow (40) may be a manual clamp, or a Robert Clip, placed anywhere along the length of the entry tube (18). The means of cutting off flow (40) may be a pinch valve integrated anywhere along the length of the entry tube (18) or integrated at the aperture (22). Further, the pinch valve may be integrated into the connector (30A) at the first end of the entry tube (18A) or integrated into the connector (30B) at the second end of the entry tube (18B).

### Pump

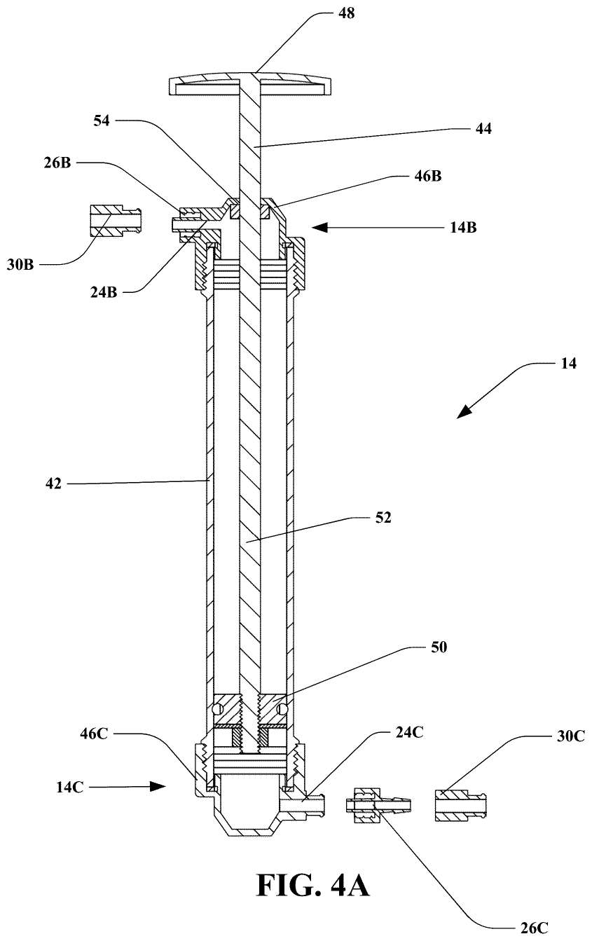

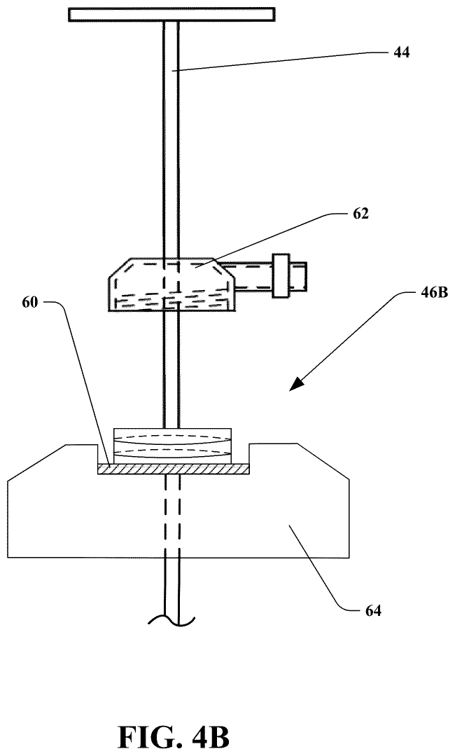

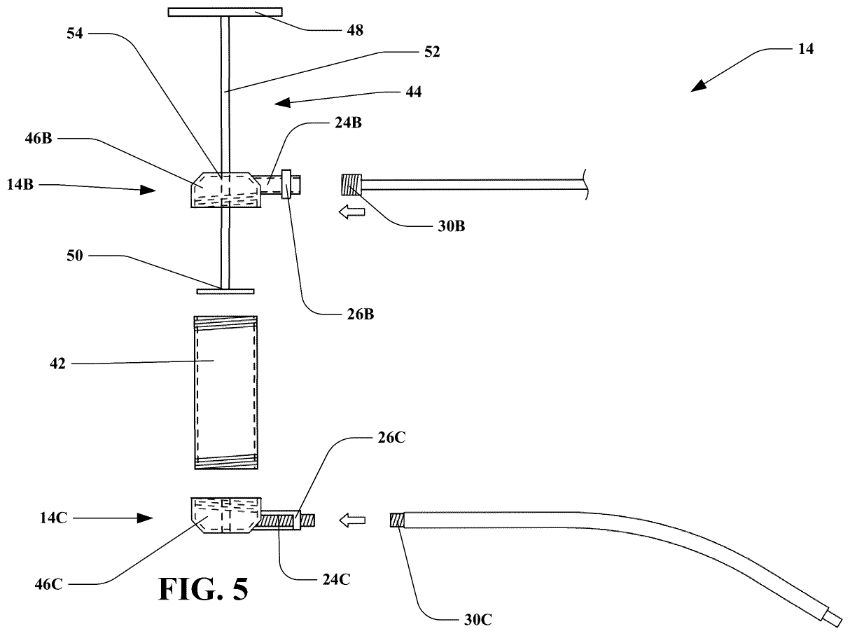

FIG. 4A shows an embodiment of a pump (14), FIG. 4B shows an alternative embodiment of an entry end (14B) and FIG. 5 shows an exploded view of the pump (14). The pump (14) may be comprised of a body (which may have any desired cross sectional shape, and is preferably in the form of a barrel; throughout the remainder of the text, the term “barrel” is used, but is not intended to specify any particular form or shape) (42) and a means of pumping (44). The barrel (42) may include markings or other appropriate method of measuring the volume of feed in the barrel (42). The barrel (42) may have an entry end (14B) and an exit end (14C), such that the entry end (14B) is defined by feed entering the barrel (42) via the first opening (24B) and the exit end is defined by feed exiting the barrel (42) via the second opening (24C). The barrel (42) may be considerably cylindrical like a barrel shape; however, any suitable shape may be contemplated. It is beneficial to have the barrel (42) hold a large amount of feed to allow the user to deliver feed to the patient efficiently. In order to improve safety, the barrel (42) volume may not be so large that if the pump (14) is dropped the force will remove a patient's feeding port (16) or otherwise injure the patient. Thus, in one embodiment, the barrel (42) may hold 40 mL to 80 mL of feed; the volume may be determined by patient's feeding specifications, patient's size, and patient feeding port-type (16). The entry end (14B) and exit end (14C) may have an entry cap (46B) and an exit cap (46C), respectively. The entry cap (46B) and the exit cap (46C) may have the first opening (24B) and the second opening (24C) integrated into each, respectively. The entry cap (46B) and the exit cap (46C) may be removably attached to the barrel (42), such that the attachment is substantially airtight. The attachment means for the cap (46B, 46C) may be threaded like a screw-type connection, a fitted connection sealed with friction, a snapped connection with tabs or grooves that snap into a rim, a latched connection to press the cap (46B, 46C) and barrel (42) together made air tight with a gasket or O-ring (60), or any other suitable means known by a person having skill in the art. The entry end (14B) and exit end (14C) is preferably configured such that all connections are airtight, to avoid air intrusion into the feed, thus avoiding introduction of air into the patient's stomach/digestive system. It should be appreciated that the pump (14) may have either the entry cap (46B) or the exit cap (46C), or the pump may have both the entry cap (46B) and the exit cap (46C), or the pump may have neither either the entry cap (46B) or the exit cap (46C) that are removable. In another embodiment, the entry cap (46B) may be made up of a first cap (62) and a second cap (64). The first cap (62) and the second cap (64) may have an airtight connection with an O-ring (60) or any other suitable means. In one embodiment, the barrel (42), entry cap (46B), exit cap (46C), connector (30A), and/or connector (30B) can be configured as a unitary assembly. This can be done, for example, by 3D printing of the assembly or any subset of the assembly components. In one embodiment, it is preferable to have both the entry cap (46B) and the exit cap (46C) to make the barrel portion (42) accessible to aid cleaning the feeding pump system (10) between feeds or daily.

The pump (14) may have a means of pumping (44) or a means of actuating the feed to flow from the feed reservoir (12) to the patient via the feeding port (16). The means of pumping (44) may be a piston-style hand pump with a plunger, a rotary hand pump, a bulb or squeeze-able body that can create suction, or any other suitable means known by a person having skill in the art. In one embodiment, a preferred means of pumping (44) is a piston-style hand pump (14) as described above, further comprising a handle (48), a plunger (50), each on opposing ends of a stem (52). The handle (48) may be any suitable material and any suitable shape, preferably optimized for comfort, efficiency, and safety during use. The stem (52) may be an elongated member of rigid material, preferably of a medical-grade, anti-microbial material. The handle (48) may be fixedly attached to one end of the stem (52). In an alternative embodiment, the handle (48) may be removably attached to the stem (52) by threaded, screw-type means, a snap on connection with a ridge and groove, or any other suitable connection. The entry end (14B) or exit end (14B) may have a hole (54) where the stem (52) is set through. The hole (54) may allow the stem (52) to move transaxially, but may be substantially sealed with a gasket or similar rubber coating to prevent leakage.

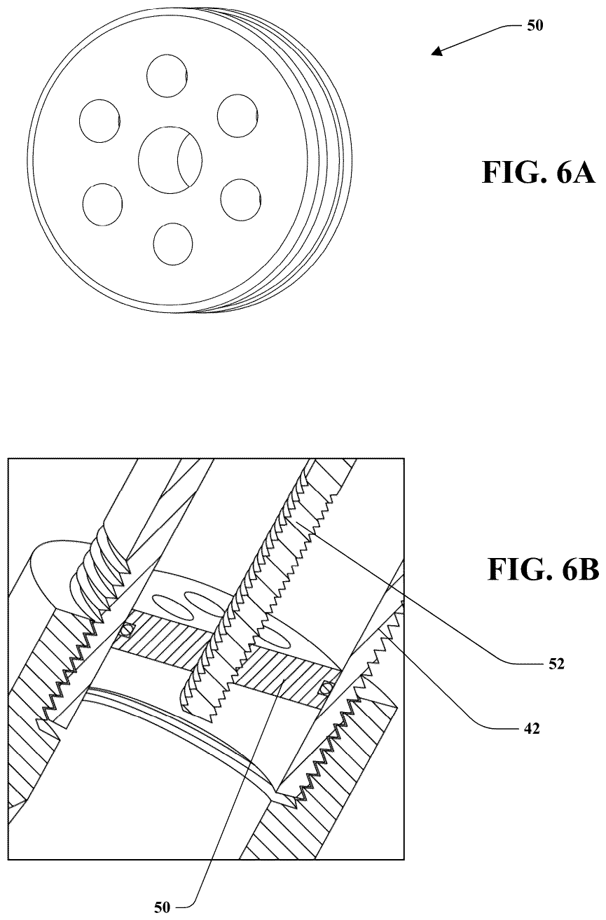

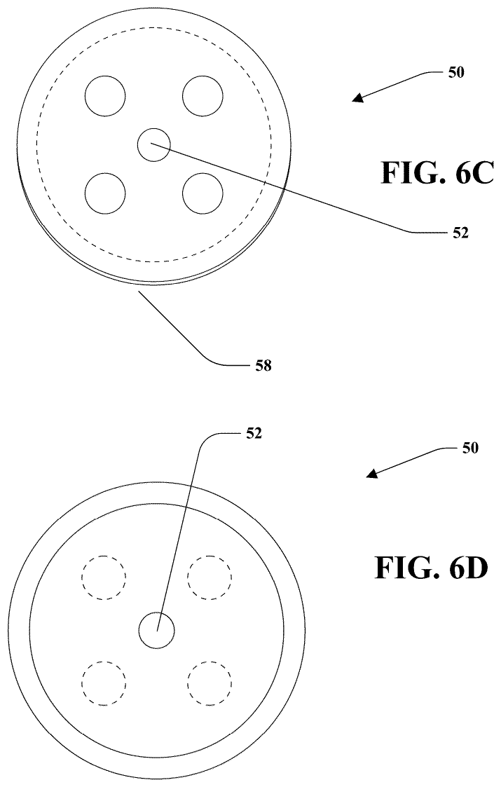

In addition to the above figures, FIGS. 6A, 6B, 6C, and 6D show detailed views of the plunger (50). The plunger (50) may be fixedly attached to one end of the stem (52), opposing the handle (48). In an alternative embodiment, the plunger (50) may be removably attached to the stem (52) by threaded, screw-type means, a snap on connection with a ridge and groove, or any other suitable connection. The plunger (50) may be made of rigid materials, preferably of a medical-grade, anti-microbial material. The plunger (50) may be configured to conformably fit inside the barrel (42) such that the diaphragm may move transaxially with some friction between the edge of the plunger (50) and the inner wall of the body (42).

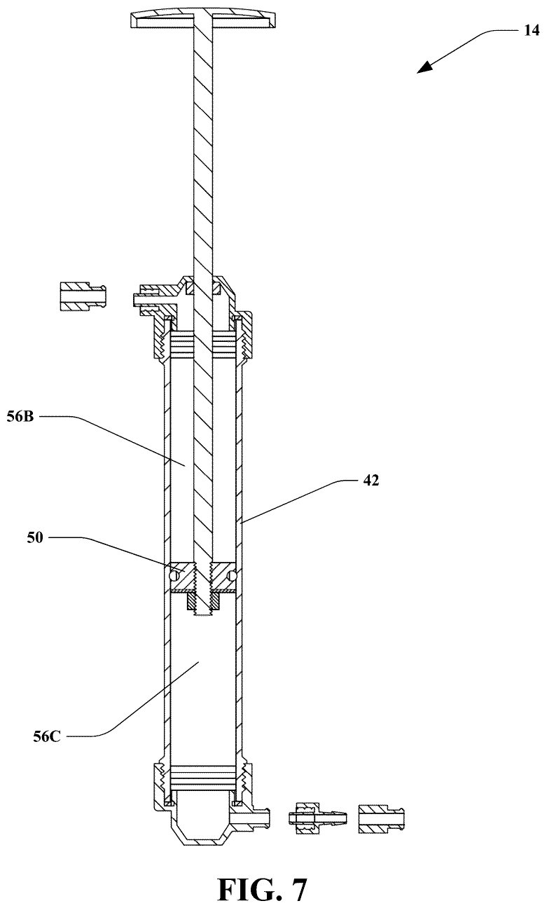

In addition to the above figures, FIG. 7 shows an embodiment of the pump (14). In operation, the plunger (50) divides the barrel (42) into an entry chamber (56B) and an exit chamber (56C), where the plunger (50) at the edges substantially creates a seal between the entry chamber (56B) and the exit chamber (56C) with a gasket or similar rubber coating to prevent leakage. The entry chamber (56B) and exit chamber (56C) may vary in volume as the diaphragm moves transaxially through the barrel (42). In one embodiment, the plunger (50) may be a disc sized to fit inside a substantially cylindrical barrel-shaped barrel (42). The plunger (50) further comprises at least one diaphragm valve (58), or any other suitable one-way valve. Feed may move from the entry chamber (56B) through the plunger (50) via the at least one diaphragm valve (58) into the exit chamber (56C), but feed may not move from the exit chamber (56C) into the entry chamber (56B).

### Priming Stage

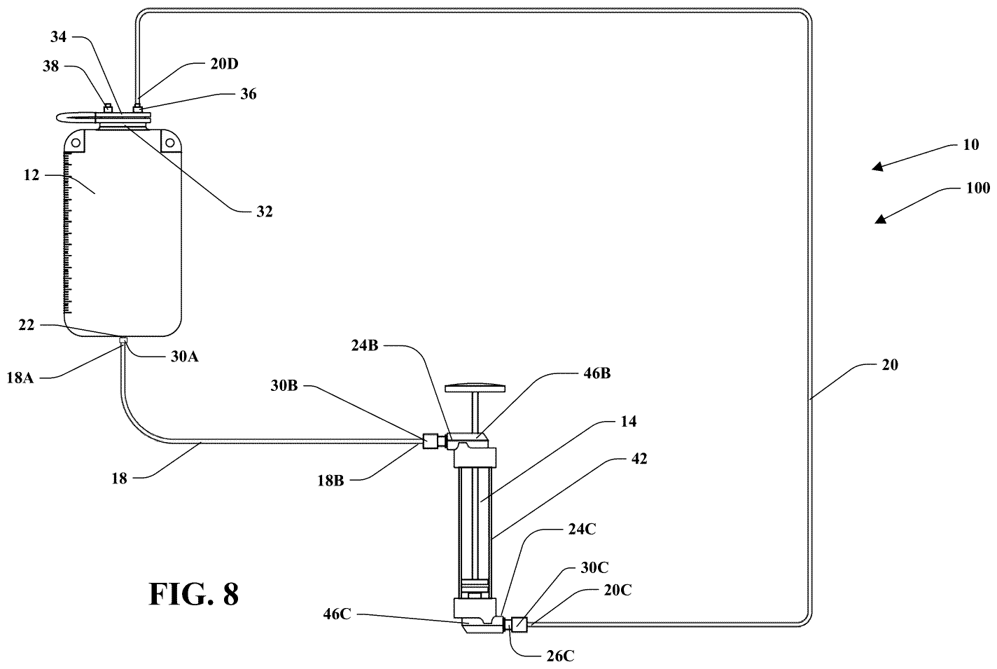

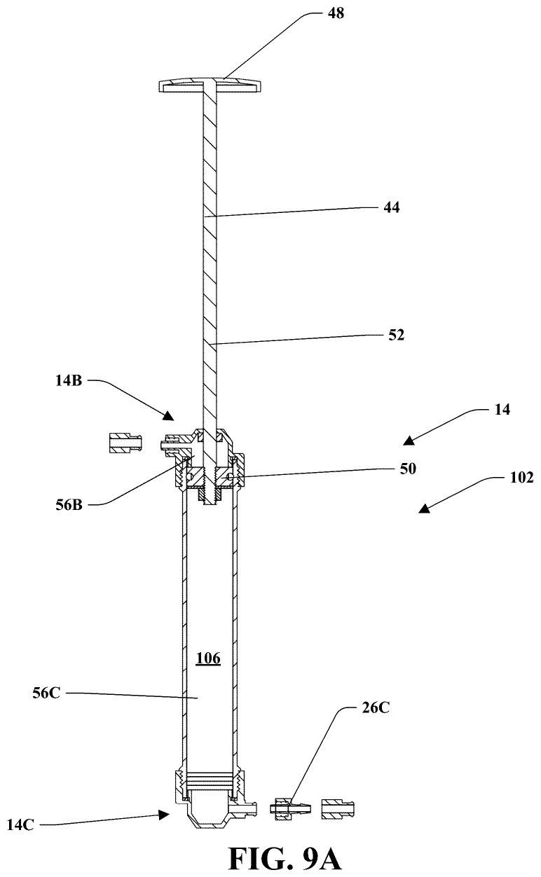

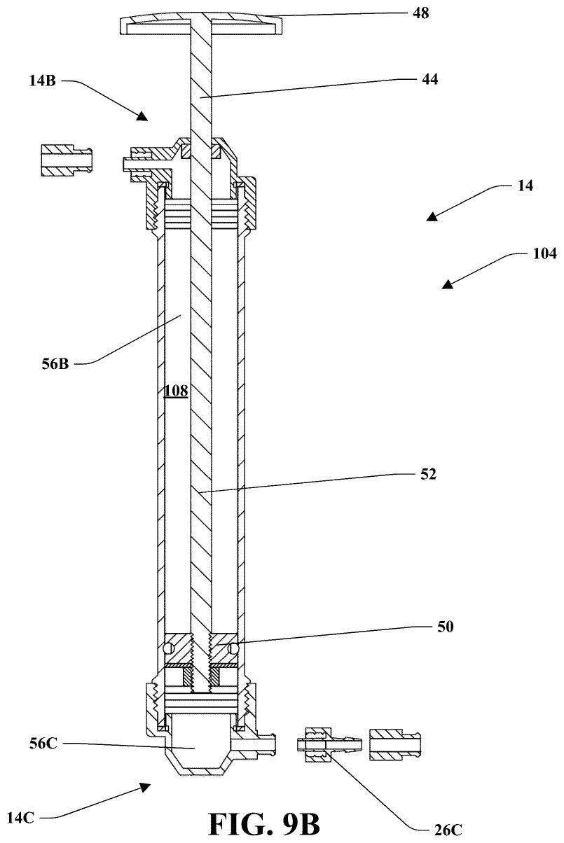

FIG. 8 shows an embodiment of the feeding pump system (10) in a priming stage (100). FIGS. 9A and 9B show the pump (14) in a first position (102) and a second position (104), respectively. In preparation for feeding a patient, the feeding pump system (10) may be primed (100) by evacuating substantially all the air from the feeding pump system (10). The feed reservoir (12) may be filled through the mouth (32) with feed to the desired amount, preferably with a patient's specified daily allotment. In another embodiment, the feed reservoir (12) may be pre-filled or have been previously filled in preparation for a patient's subsequent feeding of the day. Before priming (100), the user may ensure the lid (34) is coupled securely to the mouth (32), the first end of the entry tube (18A) is coupled with the connector (30A) to the aperture (22), the second end of the entry tube (18B) is coupled with the connector (30B) to the first opening (24B), the entry cap (46B) is coupled to the barrel (42), the exit cap (46C) is coupled to the barrel (42), the second opening (24C) is coupled with the connector (30C) to the first end of the exit tube (20C). It may be appreciated in alternative embodiments that the entry tube (18), exit tube (20), entry cap (46B), or exit cap (46C) may be fully integrated into their coupled components, respectively. The second end of the exit tube (20D) may be coupled to the feedback port (36), thus forming a closed system. It is likely upon assembly of the feeding pump system (10) for the priming stage (100) that air will be trapped in the tubing (18, 20) and in the pump (14).

To begin the priming stage (100) the means of cut off (40) may be disengaged to allow flow of the feed. The priming stage (100) may comprise the pump (14) being actuated by moving from a first position (102) to a second position (104), and repeating. In one embodiment, a preferred means of pumping (44) is a piston-style hand pump (14) as described above. The pump (14) may start in the first position (102), where the handle (48) via the stem (52) has fully pressed the plunger (50) toward the exit end (14C), with the result that the entry chamber (56B) has a lesser volume than the exit chamber (56C). The pump (14) is actuated by pulling the plunger (50) via the stem (52) with the handle (48) transaxially away from the exit end (14C) toward the entry end (14B). The pump (14) may stop in the second position (104), where the handle (48) via the stem (52) has fully pulled the plunger (50) toward the entry end (14B), with the result that the entry chamber (56B) has a greater volume than the exit chamber (56C). As the pump (14) is actuated from the first position (102) to the second position (104), a vacuum (106) is created in the exit chamber (56C). The exit chamber vacuum (106) forces air and/or feed to move from the feed reservoir (12) into the entry chamber (56B) through the plunger (50) into the exit chamber (56C). It may be appreciated that the second valve (26C) prevents the exit chamber vacuum (106) from drawing air and/or feed from the exit tube (20) into the exit chamber (56C). Then, the pump (14) may be moved from the second position (104) to the first position (102), where the handle (48) via the stem (52) presses the plunger (50) toward the exit end (14C). The pump (14) may stop in the first position (102), where the handle (48) via the stem (52) has fully pressed the plunger (50) toward the exit end (14C). As the pump (14) is actuated from the second position (104) to the first position (102), a vacuum (108) is created in the entry chamber (56B). The entry chamber vacuum (108) forces air and/or feed to move from the feed reservoir (12) into the entry chamber (56B), while the plunger (50) presses air and/or feed out of the exit chamber (56C) and into the feed reservoir (12) via the feedback port (36). It may be appreciated that the at least one diaphragm valve (58) prevents the entry chamber vacuum (108) from drawing air and/or feed from the exit chamber (56C) into the entry chamber (56B). The priming stage (100) continues, moving from the first position (102) to the second position (104) and back, while the air is pressed out of the air release valve (38) and feed returns to the feed reservoir (12) until substantially all the air is evacuated form the feeding pump system (10). This may be determined by priming (100) until feed is pressed out of the air release valve, indicating that no air remains. It may be appreciated that the priming stage (100) may use gravity to move air upward toward the air release valve (38), thus the air release valve (38) may be oriented at the to be at the top of the feed reservoir (12) in order to allow substantially all of the air to evacuate. To conclude the priming stage (100) the means of cut off (40) may be returned to cease flow of the feed.

### Feeding Stage

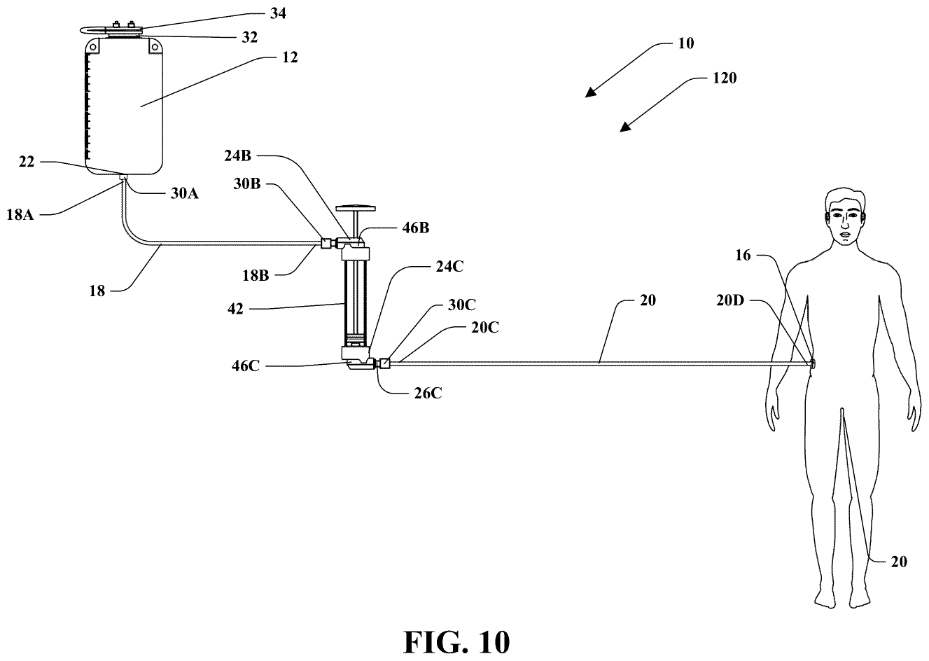

FIG. 10 shows an embodiment of the feeding pump system (10) in a feeding stage (120). It is preferable to have substantially all the air evacuated from the feeding pump system (10) before the feeding stage (120). Before feeding (120), the user may ensure the lid (34) is coupled to the mouth (32), the first end of the entry tube (18A) is coupled with the connector (30A) to the aperture (22), the second end of the entry tube (18B) is coupled with the connector (30B) to the first opening (24B), the entry cap (46B) is coupled to the barrel (42), the exit cap (46C) is coupled to the barrel (42), the second opening (24C) is coupled with the connector (30C) to the first end of the exit tube (20C). It may be appreciated in alternative embodiments that the entry tube (18), exit tube (20), entry cap (46B), or exit cap (46C) may be fully integrated into their coupled components, respectively. The second end of the exit tube (20D) may be coupled to the patient's feeding port (16).

To begin the feeding stage (120) the means of cut off (40) may be disengaged to allow flow of the feed. The feeding stage (120) may comprise the pump (14) being actuated by moving from the first position (102) to the second position (104), and repeating. In one embodiment, a preferred means of pumping (44) is a piston-style hand pump (14) as described above. The pump (14) may start in the first position (102), where the handle (48) via the stem (52) has fully pressed the plunger (50) toward the exit end (14C), with the result that the entry chamber (56B) has a lesser volume than the exit chamber (56C). The pump (14) is actuated by pulling the plunger (50) via the stem (52) with the handle (48) transaxially away from the exit end (14C) toward the entry end (14B). The pump (14) may stop in the second position (104), where the handle (48) via the stem (52) has fully pulled the plunger (50) toward the entry end (14B), with the result that the entry chamber (56B) has a greater volume than the exit chamber (56C). As the pump (14) is actuated from the first position (102) to the second position (104), a vacuum (106) is created in the exit chamber (56C). The exit chamber vacuum (106) forces feed to move from the feed reservoir (12) into the entry chamber (56B) through the plunger (50) into the exit chamber (56C). It may be appreciated that the second valve (26C) prevents the exit chamber vacuum (106) from drawing feed or patient's stomach contents from the exit tube (20) into the exit chamber (56C). Then, the pump (14) may be moved from the second position (104) to the first position (102), where the handle (48) via the stem (52) presses the plunger (50) toward the exit end (14C). The pump (14) may stop in the first position (102), where the handle (48) via the stem (52) has fully pressed the plunger (50) toward the exit end (14C). As the pump (14) is actuated from the second position (104) to the first position (102), a vacuum (108) is created in the entry chamber (56B). The entry chamber vacuum (108) forces feed to move from the feed reservoir (12) into the entry chamber (56B), while the plunger (50) presses feed out of the exit chamber (56C) and into the patient via the feeding port (16). It may be appreciated that the at least one diaphragm valve (58) prevents the entry chamber vacuum (108) from drawing feed from the exit chamber (56C) into the entry chamber (56B). The feeding stage (120) continues, moving from the first position (102) to the second position (104) and back, until patient satisfaction or until the feed reservoir (12) has been depleted. The rate of actuating the pump (14) may be a factor to contribute to the rate of feeding. It may be appreciated that the feeding stage (120) may not require gravity to move feed into the patient. To conclude the feeding stage (120) the means of cut off (40) may be reengaged to cease flow of the feed.

During the feeding stage or during transport, the feed reservoir (12) and/or the pump (14) may be housed in a system of straps. The system of straps may prevent the feed reservoir (12) or pump (14) from falling and tugging on the patient's port. The system of straps may be made of neoprene, rubber, silicone, nylon, polyurethane, polypropylene, polyester, a combination of materials, or any other suitable materials. The system of straps may include loops for securing the pump (14) to the user's hand during the feeding stage.

### Cleaning Stage

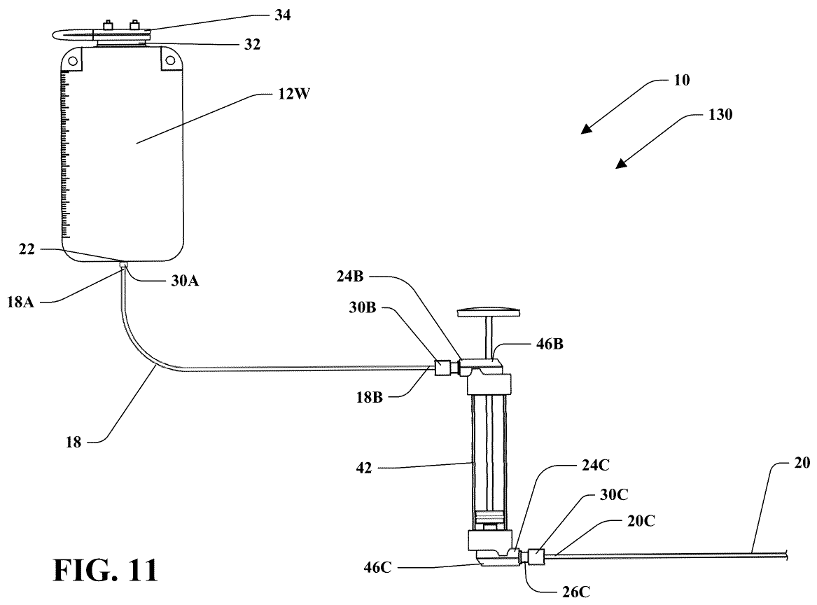

FIG. 11 shows an embodiment of the feeding pump system (10) in a cleaning stage (130). The feeding pump system (10) may be cleaned (130) after every feeding stage (120) or daily. The cleaning stage (130) may include a feeding reservoir (12W) filled with water and/or cleaning solution. “Cleaning solution” may include, but is not limited to, dish soap, disinfectant, sterilizing agents, enzymatic cleaners, or the like. Before cleaning (130), the user may ensure the lid (34) is coupled to the mouth (32), the first end of the entry tube (18A) is coupled with the connector (30A) to the aperture (22), the second end of the entry tube (18B) is coupled with the connector (30B) to the first opening (24B), the entry cap (46B) is coupled to the barrel (42), the exit cap (46C) is coupled to the barrel (42), the second opening (24C) is coupled with the connector (30C) to the first end of the exit tube (20C). It may be appreciated in alternative embodiments that the entry tube (18), exit tube (20), entry cap (46B), or exit cap (46C) may be fully integrated into their coupled components, respectively. The second end of the exit tube (20D) may be directed to a drain or a trash can, or any other suitable disposal location.

To begin the cleaning stage (130) the means of cut off (40) may be disengaged to allow the flow of water and/or cleaning solution. The cleaning stage (130) may comprise the pump (14) being actuated by moving from the first position (102) to the second position (104), and repeating. In one embodiment, a preferred means of pumping (44) is a piston-style hand pump (14) as described above, and further comprising transparent components to visually assess the feeding pump system's (10) cleanliness. The pump (14) may start in the first position (102), where the handle (48) via the stem (52) has fully pressed the plunger (50) toward the exit end (14C), with the result that the entry chamber (56B) has a lesser volume than the exit chamber (56C). The pump (14) is actuated by pulling the plunger (50) via the stem (52) with the handle (48) transaxially away from the exit end (14C) toward the entry end (14B). The pump (14) may stop in the second position (104), where the handle (48) via the stem (52) has fully pulled the plunger (50) toward the entry end (14B), with the result that the entry chamber (56B) has a greater volume than the exit chamber (56C). As the pump (14) is actuated from the first position (102) to the second position (104), a vacuum (106) is created in the exit chamber (56C). The exit chamber vacuum (106) forces water and/or cleaning solution to move from the feed reservoir (12W) into the entry chamber (56B) through the plunger (50) into the exit chamber (56C). It may be appreciated that the second valve (26C) prevents the exit chamber vacuum (106) from drawing residual feed, water and/or cleaning solution from the exit tube (20) into the exit chamber (56C). Then, the pump (14) may be moved from the second position (104) to the first position (102), where the handle (48) via the stem (52) presses the plunger (50) toward the exit end (14C). The pump (14) may stop in the first position (102), where the handle (48) via the stem (52) has fully pressed the plunger (50) toward the exit end (14C). As the pump (14) is actuated from the second position (104) to the first position (102), a vacuum (108) is created in the entry chamber (56B). The entry chamber vacuum (108) forces water and/or cleaning solution to move from the feed reservoir (12W) into the entry chamber (56B), while the plunger (50) presses water and/or cleaning solution out of the exit chamber (56C) and into a disposal location. It may be appreciated that the at least one diaphragm valve (58) prevents the entry chamber vacuum (108) from drawing water and/or cleaning solution from the exit chamber (56C) into the entry chamber (56B). The cleaning stage (130) continues, moving from the first position (102) to the second position (104) and back, until visually clean or until the feed reservoir (12W) has been depleted. In another embodiment, the cleaning stage (13) may include immediately flushing with water after the feeding stage (120), then repeating flushing with a water and cleaning solution until feed residual is gone, and then repeating flushing with water until the cleaning solution is gone from the feeding pump system (10). In an alternative embodiment, the cleaning stage (130) may include disassembly of the components from which they are coupled in order to mechanically clean components with a brush, sponge, or the like. It may be preferred that the feeding pump system (10) be cleaned at least once daily and disposed of or recycled after approximately one month of use.

The following are non-limiting examples of some embodiments of the present invention:

Embodiment 1. A pump for use in a system for tube feeding, the pump comprising:
a body portion having a manual plunger therein, and having a first opening with a first valve and a second opening with a second valve; wherein the first opening with the first valve is configured to permit flow of a liquid feed composition through the first opening into the barrel portion;
wherein the second opening with the second valve is configured to permit flow of the liquid feed composition through the second opening out of the body portion; and
wherein the manual plunger is configured to draw the liquid feed composition through the first opening into the body portion when pulled to a withdrawn position, and to push the liquid feed composition through the second opening out of the body portion as the manual plunger is pushed into the body portion.

Embodiment 2. The pump according to Embodiment 1, further comprising:
an entry cap housing the first opening with the first valve; and
an exit cap housing the second opening with the second valve;
wherein the entry cap and the body portion are connected by an airtight seal.

Embodiment 3. The system for tube feeding according to Embodiment 2 wherein the airtight seal includes an O-ring.

Embodiment 4. A system for tube feeding, comprising:
a feed reservoir having an aperture; and
the pump according to Embodiment 1,
   wherein the feed reservoir is coupled to the first opening of the pump via the aperture.

Embodiment 5. The system according to Embodiment 4, further comprising:
a feeding port housed on a patient, wherein the feed reservoir is coupled to the first opening of the pump via the aperture, and second opening of the pump is coupled to the feeding port.

Embodiment 6. The system according to Embodiment 4 further comprising:
an entry tube having a first end and a second end, wherein the aperture is coupled to the first end of the entry tube and the second end of the entry tube is coupled to the first opening of the pump.

Embodiment 7. The system for tube feeding according to Embodiment 4 wherein the entry tube size is 5 to 36 French Units.

Embodiment 8. The system for tube feeding according to any one of Embodiments 4 to 7, further comprising:
an exit tube having a first end and a second end, wherein the second opening of the pump is coupled to the first end of the exit tube and the second end of the exit tube is coupled to the feeding port.

Embodiment 9. The system for tube feeding according to Embodiment 8 wherein the exit tube size is 5 to 36 French Units.

Embodiment 10. A method of feeding with a system for tube feeding, wherein the system for tube feeding comprises:
a feed reservoir having an aperture;
   a pump having a first opening with a first valve configured to flow into the pump, and a second opening with a second valve configured to flow out of the pump; and
   a feeding port housed on a patient, wherein the feed reservoir is coupled to the first opening of the pump via the aperture, and second opening of the pump is coupled to the feeding port, the method comprising:
   pumping the air out of the system;
   drawing feed out of the feed reservoir into the pump through the first opening having the first valve; and
   pushing feed out of the pump through the second opening having the second valve, and through the feeding port into the patient.

Embodiment 11. A pump for use in a system for tube feeding, the pump comprising:
a body portion having a manual plunger therein and
a diaphragm valve;
wherein a first opening is configured to permit flow of a liquid feed composition through the first opening into the body portion;
   wherein a second opening is configured to permit flow of the liquid feed composition through the second opening out of the body portion; and
   wherein the manual plunger is configured to draw the liquid feed composition through the first opening into the body portion when pulled to a withdrawn position, and to push the liquid feed composition through the second opening out of the body portion as the manual plunger is pushed into the body portion.

Embodiment 12. The pump according to Embodiment 11, wherein the first opening has a first valve.

Embodiment 13. The pump according to Embodiment 11 or Embodiment 12, wherein the second opening has a second valve.

Embodiment 14. The pump according to Embodiment 12 or Embodiment 13, wherein the first valve is a one-way valve.

Embodiment 15. The pump according to Embodiment 13 or Embodiment 14, wherein the second valve is a one-way valve.

Embodiment 16. The pump according to any one of Embodiments 11 to 15, further comprising an entry cap housing the first opening.

Embodiment 17. The pump according to Embodiment 16, wherein the entry cap and the body portion are connected by an airtight seal.

Embodiment 18. The pump according to Embodiment 17, wherein the airtight seal includes an O-ring.

Embodiment 19. The pump according to any one of Embodiments 11 to 18, further comprising an exit cap housing the second opening.

Embodiment 20. The pump of any one of Embodiments 11 to 19, wherein the body portion is cylindrical.

The present application is directed to human medical applications, but may also be altered to be applicable in veterinary sciences. Although certain embodiments have been shown and described, it is understood that equivalents and modifications falling within the scope of the appended claims will occur to others who are skilled in the art upon the reading and understanding of this specification.

What is claimed is:

1. A pump for use in a system for tube feeding, the pump comprising:
a body portion having a manual plunger therein, and having a first opening with a first valve and a second opening with a second valve;
wherein the first opening with the first valve is configured to permit flow of a liquid feed composition through the first opening into the barrel portion;
wherein the second opening with the second valve is configured to permit flow of the liquid feed composition through the second opening out of the body portion; and
wherein the manual plunger is configured to draw the liquid feed composition through the first opening into the body portion when pulled to a withdrawn position, and to push the liquid feed composition through the second opening out of the body portion as the manual plunger is pushed into the body portion.

2. The pump of claim 1, further comprising:
an entry cap housing the first opening with the first valve; and
an exit cap housing the second opening with the second valve;
wherein the entry cap and the body portion are connected by an airtight seal.

3. The system for tube feeding of claim 2 wherein the airtight seal includes an O-ring.

4. A system for tube feeding, comprising:
a feed reservoir having an aperture; and
the pump according to claim 1,
wherein the feed reservoir is coupled to the first opening of the pump via the aperture.

5. The system for tube feeding of claim 4, further comprising:
a feeding port housed on a patient, wherein the feed reservoir is coupled to the first opening of the pump via the aperture, and second opening of the pump is coupled to the feeding port.

6. The system for tube feeding according to claim 4 further comprising:
an entry tube having a first end and a second end, wherein the aperture is coupled to the first end of the entry tube and the second end of the entry tube is coupled to the first opening of the pump.

7. The system for tube feeding according to claim 4 wherein the entry tube size is 5 to 36 French Units.

8. The system for tube feeding according to claim 5 further comprising:
an exit tube having a first end and a second end, wherein the second opening of the pump is coupled to the first end of the exit tube and the second end of the exit tube is coupled to the feeding port.

9. The system for tube feeding according to claim 8 wherein the exit tube size is 5 to 36 French Units.

10. A method of feeding with a system for tube feeding, wherein the system for tube feeding comprises:
a feed reservoir having an aperture;
a pump having a first opening with a first valve configured to flow into the pump, and a second opening with a second valve configured to flow out of the pump; and
a feeding port housed on a patient, wherein the feed reservoir is coupled to the first opening of the pump via the aperture, and second opening of the pump is coupled to the feeding port, the method comprising:
pumping the air out of the system;
drawing feed out of the feed reservoir into the pump through the first opening having the first valve; and
pushing feed out of the pump through the second opening having the second valve, and through the feeding port into the patient.

11. A pump for use in a system for tube feeding, the pump comprising:
a body portion having a manual plunger therein and
a diaphragm valve;
wherein a first opening is configured to permit flow of a liquid feed composition through the first opening into the body portion;
wherein a second opening is configured to permit flow of the liquid feed composition through the second opening out of the body portion; and
wherein the manual plunger is configured to draw the liquid feed composition through the first opening into the body portion when pulled to a withdrawn position, and to push the liquid feed composition through the second opening out of the body portion as the manual plunger is pushed into the body portion.

12. The pump according to claim 11, wherein the first opening has a first valve.

13. The pump according to claim 11, wherein the second opening has a second valve.

14. The pump according to claim 12, wherein the first valve is a one-way valve.

15. The pump according to claim 13, wherein the second valve is a one-way valve.

16. The pump according to claim 11, further comprising an entry cap housing the first opening.

17. The pump according to claim 16, wherein the entry cap and the body portion are connected by an airtight seal.

18. The pump according to claim 17, wherein the airtight seal includes an O-ring.

19. The pump according to claim 11, further comprising an exit cap housing the second opening.

20. The pump of claim 11, wherein the body portion is cylindrical.
